(19) United States
(12) Patent Application Publication
(10) Pub. No.: US 2026/0325346 A1
(43) Pub. Date: Oct. 22, 2026

(54) HEATED GARMENT FOR CHEMOTHERAPY TREATMENTS AND METHOD THEREFOR

(71) Applicant: Akena Mosley, Burbank, CA (US)

(72) Inventor: Akena Mosley, Burbank, CA (US)

(21) Appl. No.: 19/181,578

(22) Filed: Apr. 17, 2025

(51) Int. Cl. A61F 7/08; A41D 13/005; A41D 13/12; A61F 7/00; A61M 5/14

(52) U.S. Cl. CPC A61F 7/08; A41D 13/0051; A41D 13/1245; A61F 7/007; A61M 5/1415; A41D 2400/12; A41D 2400/32; A61F 2007/008; A61M 2005/1416

(57) ABSTRACT

A heated garment for chemotherapy treatments includes a torso portion, an access panel, and a heating assembly. The torso portion includes a neck opening and arm openings. The torso portion is designed to be worn on a torso. The torso portion has a front side and a back side. The torso portion includes an aperture in the front side for insertion of medical tubing through the torso portion. The access panel is coupled to the torso portion over the aperture. The access panel is at least partially detachable from the torso portion to at least partially uncover the aperture for insertion of the medical tubing. The heating assembly is coupled to the torso portion. The heating assembly is designed to be selectively activated to heat the torso.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to heated jackets and shirts for chemotherapy and more particularly pertains to a new heated garment for keeping a patient warm during chemotherapy treatments. Current shirts and hoodies are required to be mostly or completely open in front in order to connect the medical tubing from the supply of chemotherapy drugs to the patient, which leaves the patient's chest exposed and the patient being cold. The new heated garment allows connection of the medical tubing through the heated garment so the garment can stay closed. In addition, the new heated garment includes a heating assembly to actively warm the patient. (2) Description of Related Art including information disclosed under 37 CFR 1.97 and 1.98.

The prior art relates to heated jackets and shirts for chemotherapy. The prior art, as best understood, does not disclose a heated garment for chemotherapy treatments that includes a torso portion, an access panel, and a heating assembly, wherein the torso portion includes an aperture in the front side for insertion of medical tubing through the torso portion without having to open the heated garment in the front or the rear.

### (h) BRIEF SUMMARY OF THE INVENTION

An embodiment of the disclosure meets the needs presented above in a heated garment generally comprising a torso portion, an access panel, and a heating assembly. The torso portion includes a neck opening and arm openings. The torso portion is designed to be worn on a torso. The torso portion has a front side and a back side. The torso portion includes an aperture in the front side for insertion of medical tubing through the torso portion. The access panel is coupled to the torso portion over the aperture. The access panel is at least partially detachable from the torso portion to at least partially uncover the aperture for insertion of the medical tubing. The heating assembly is coupled to the torso portion. The heating assembly is designed to be selectively activated to heat the torso.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a heated garment according to an embodiment of the disclosure.

FIG. 2 is a rear view of an embodiment of the disclosure.

FIG. 3 is a front view of an embodiment of the disclosure.

FIG. 4 is a front view of an embodiment of the disclosure in use.

### (j) DETAILED DESCRIPTION OF THE INVENTION

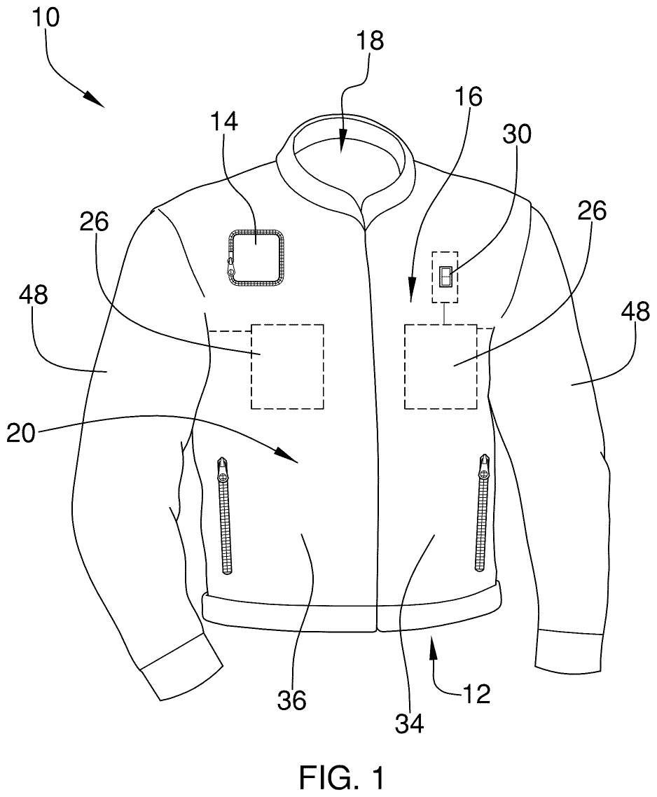

With reference now to the drawings, and in particular to FIGS. 1 through 4 thereof, a new heated garment embodying the principles and concepts of an embodiment of the disclosure and generally designated by the reference numeral 10 will be described.

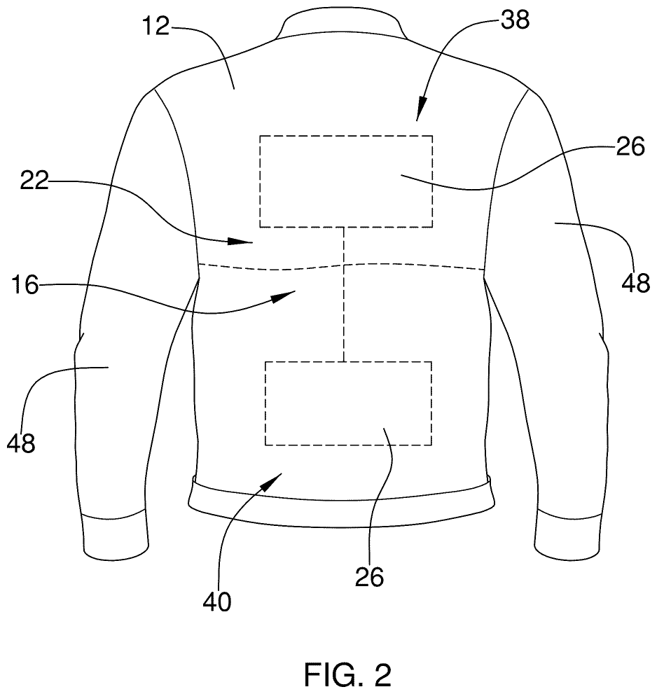

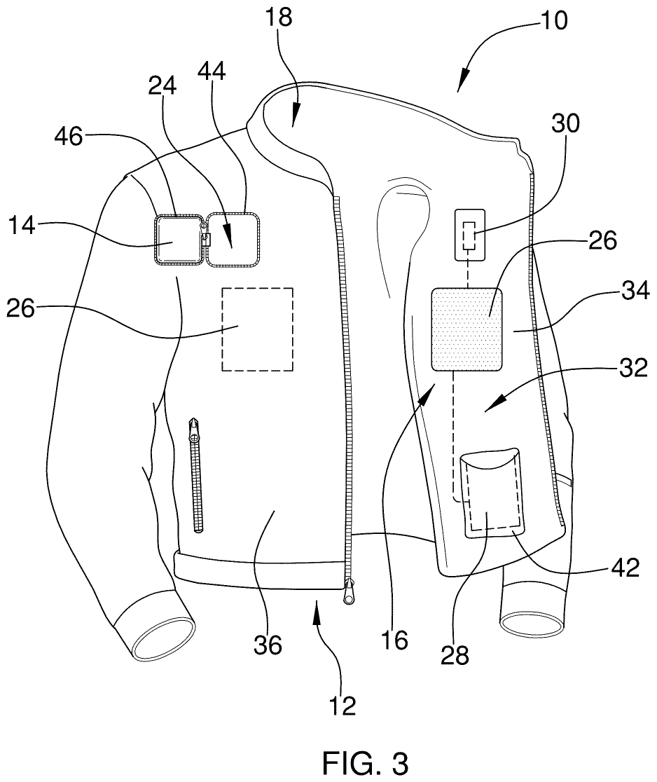

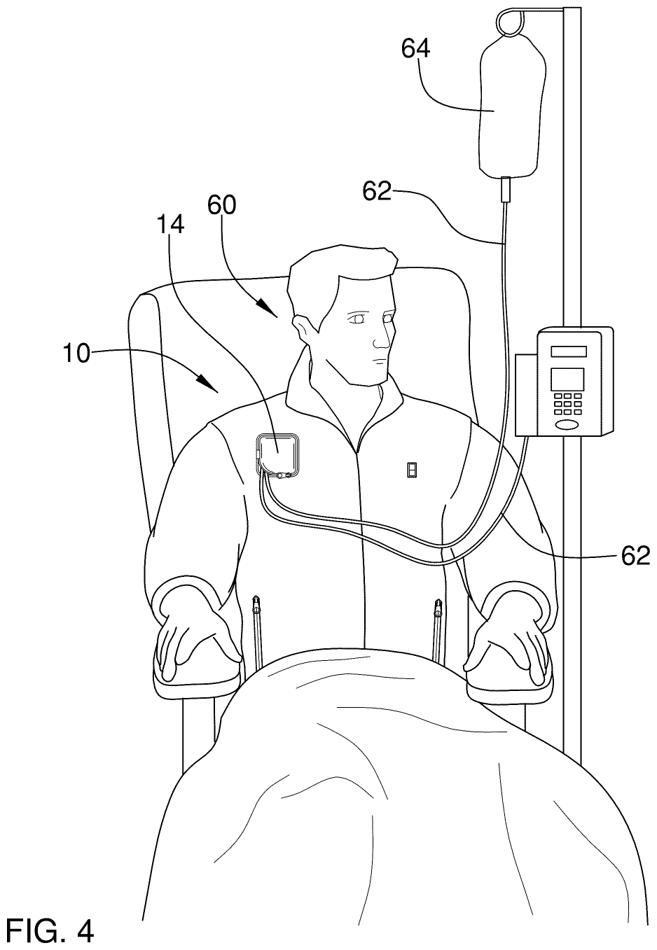

As best illustrated in FIGS. 1 through 4, the heated garment 10 for chemotherapy treatments generally comprises a torso portion 12, an access panel 14, and a heating assembly 16. The torso portion 12 includes a neck opening 18 and arm openings (not shown). The torso portion 12 is designed to be worn on a torso, as shown in FIG. 4. The torso portion 12 has a front side 20 and a back side 22, as shown in FIGS. 1 and 2, respectively. As best seen in FIG. 3, the torso portion 12 includes an aperture 24 in the front side 20 for insertion of medical tubing 62 through the torso portion 12. The access panel 14 is coupled to the torso portion 12 over the aperture 24, as shown in FIG. 1. The access panel 14 is at least partially detachable from the torso portion 12 to at least partially uncover the aperture 24 for insertion of the medical tubing 62, as shown in FIGS. 3 and 4. The heating assembly 16 is coupled to the torso portion 12. The heating assembly 16 is designed to be selectively activated to heat the torso.

In the exemplary embodiment in FIG. 3, the heating assembly 16 includes a plurality of heating pads 26, a rechargeable battery 28, and a power switch 30. The rechargeable battery 28 is connected to the heating pads 26 to supply power to the heating pads 26. The power switch 30 is connected to the rechargeable battery 28 and the heating pads 26 to turn the heating assembly 16 on and off. In the exemplary embodiment in FIG. 3, the power switch 30 is positioned at the front side 20 of the torso portion 12. The power switch 30 could be designed as a flip switch that projects out from the front side 20, or could be designed as a pressable button that is embedded in the front side 20.

In the exemplary embodiment in FIGS. 1 through 3, at least one of the heating pads 26 is positioned at the front side 20 of the torso portion 12, and at least one of the heating pads 26 is positioned at the back side 22 of the torso portion 12. The heating pads 26 are embedded in the torso portion 12. However, in another possible embodiment, the heating pads 26 could be positioned on an interior surface 32 of the heated garment 10, such that the heating pads 26 abut against the torso of the patient 60 or clothing on the torso of the patient 60.

In the exemplary embodiment in FIG. 3, the front side 20 of the torso portion 12 includes a left flap 34 and a right flap 36 detachably coupled together, such as by a zipper or buttons or snaps, as is well known in the clothing arts. One of the heating pads 26 is positioned in each of the left flap 34 and the right flap 36. In the exemplary embodiment in FIG. 2, the rear side 22 of the torso portion 12 includes an upper back section 38 and a lower back section 40. One of the heating pads 26 is positioned in each of the upper back section 38 and the lower back section 40. It should be noted that the embodiment in FIGS. 1 through 3 is only one possible embodiment, and in other possible embodiments there could be more or fewer heating pads 26. In addition, the heating pads 26 could be of different designs and shapes, such as elongated strips or circular, as opposed to the square or rectangular design in the figures.

The heated garment 10 further includes a storage pocket 42. The storage pocket 42 is coupled to the interior surface 32 of the torso portion 12. The rechargeable battery 28 is stored in the storage pocket 42. While the exemplary embodiment includes the rechargeable battery 28 to power the heating pads 26, in another possible embodiment, the rechargeable battery 28 could be omitted and the heating pads 26 could be replaceable chemical heating packets insertable into corresponding pockets or slots in the torso portion 12.

The access panel 14 is coupled to the torso portion 12 by a zipper 44 that extends around a perimeter edge 46 of the access panel 14 such that the access panel 14 is detachable to completely uncover the aperture 24. Other connections, such as buttons or snaps, could be used instead of the zipper 44.

The access panel 14 is positioned adjacent the neck opening 18. However, the access panel 14 could be positioned lower on the heated garment 10.

The heated garment 10 further includes a pair of sleeves 48 that are attached to the torso portion 12. The heated garment 10 is therefore in the form of a jacket or coat, though it could also be designed as a hoodie or sweatshirt or sweater, or possibly a short-sleeve shirt, such as a T-shirt or golf shirt. In one possible embodiment, the sleeves 48 could be omitted and the heated garment 10 would be sleeveless like a vest, tank top, or similar clothing article. Virtually any clothing design is within the scope of the disclosure. In another possible embodiment, one or more of the heating pads 26 could be positioned in the sleeves 48.

The torso portion 12 and sleeves 48 can be of any well-known material used for clothing, especially warming or insulating materials, such as fleece, wool, cotton, polyester, and combinations or blends thereof.

As mentioned above, the heated garment 10 is to be used to keep the patient 60 warm during chemotherapy treatments. The patient 60 wears the garment 10 over his or her torso. The access panel 14 is then at least partially opened. Medical tubing 62, which is connected to a supply 64 of chemotherapy medication, is then inserted through the aperture 24 and connected to the patient 60 intravenously or via a port in the patient 60. The heating assembly 16 is activated to warm the patient 60.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated garment for chemotherapy treatments comprising:
a torso portion, the torso portion including a neck opening and arm openings, the torso portion being configured to be worn on a torso, the torso portion having a front side and a back side, the torso portion including an aperture in the front side for insertion of medical tubing through the torso portion;
an access panel, the access panel being coupled to the torso portion over the aperture, the access panel being at least partially detachable from the torso portion to at least partially uncover the aperture for insertion of the medical tubing; and
a heating assembly, the heating assembly being coupled to the torso portion, the heating assembly being configured to be selectively activated to heat the torso.

2. The heated garment of claim 1, wherein the heating assembly includes:
a plurality of heating pads;
a rechargeable battery connected to the heating pads to supply power to the heating pads; and
a power switch connected to the battery and the heating pads to turn the heating assembly on and off.

3. The heated garment of claim 2, wherein the power switch is positioned at the front side of the torso portion.

4. The heated garment of claim 2, wherein:
at least one of the heating pads is positioned at the front side of the torso portion; and
at least one of the heating pads is positioned at the back side of the torso portion.

5. The heated garment of claim 4, wherein:
the front side of the torso portion includes a left flap and a right flap detachably coupled together; and
one of the heating pads is positioned in each of the left flap and the right flap.

6. The heated garment of claim 2, wherein:
the rear side of the torso portion includes an upper back section and a lower back section; and
one of the heating pads is positioned in each of the upper back section and the lower back section.

7. The heated garment of claim 2, wherein the heating pads are embedded in the torso portion.

8. The heated garment of claim 1, wherein the access panel is coupled to the torso portion by a zipper that extends around a perimeter edge of the access panel such that the access panel is detachable to completely uncover the aperture.

9. The heated garment of claim 1, wherein the access panel is positioned adjacent the neck opening.

10. The heated garment of claim 1, further comprising a storage pocket, the storage pocket being coupled to an interior surface of the torso portion, the rechargeable battery being stored in the storage pocket.

11. The heated garment of claim 1, further comprising a pair of sleeves being attached to the torso portion.

12. The heated garment of claim 4, wherein:
the rear side of the torso portion includes an upper back section and a lower back section; and
one of the heating pads is positioned in each of the upper back section and the lower back section.

13. The heated garment of claim 12, wherein:
the front side of the torso portion includes a left flap and a right flap detachably coupled together; and
one of the heating pads is positioned in each of the left flap and the right flap.

14. The heated garment of claim 13, wherein the heating pads are embedded in the torso portion.

15. The heated garment of claim 14, wherein the access panel is coupled to the torso portion by a zipper that extends around a perimeter edge of the access panel such that the access panel is detachable to completely uncover the aperture.

16. The heated garment of claim 15, further comprising a storage pocket, the storage pocket being coupled to an interior surface of the torso portion, the rechargeable battery being stored in the storage pocket.

17. The heated garment of claim 16, further comprising a pair of sleeves being attached to the torso portion.

18. The heated garment of claim 17, wherein the access panel is positioned adjacent the neck opening.

19. The heated garment of claim 18, wherein the power switch is positioned at the front side of the torso portion.

20. A method of keeping a patient warm during chemotherapy treatments, the method comprising the steps of:
providing a heated garment comprising:
a torso portion, the torso portion including a neck opening and arm openings, the torso portion being configured to be worn on a torso, the torso portion having a front side and a back side, the torso portion including an aperture in the front side for insertion of medical tubing through the torso portion;
an access panel, the access panel being coupled to the torso portion over the aperture, the access panel being at least partially detachable from the torso portion to at least partially uncover the aperture for insertion of the medical tubing; and
a heating assembly, the heating assembly being coupled to the torso portion, the heating assembly being configured to be selectively activated to heat the torso;
placing the garment over a torso of a patient;
at least partially opening the access panel;
inserting medical tubing connected to a supply of chemotherapy medication through the aperture;
connecting the medical tubing intravenously or to a port in the patient; and
activating the heating assembly to warm the patient.
